(19) United States
(12) Patent Application Publication
(10) Pub. No.: US 2008/0293640 A1
(43) Pub. Date: Nov. 27, 2008

(54) Polypeptide inhibitors of HSP27 kinase and uses therefor

(75) Inventors: Colleen Brophy, Tempe, AZ (US); Alyssa Panitch, W. Lafayette, IN (US); Brandon Seal, Mesa, AZ (US); Padmini Komalavilas, Tempe, AZ (US); Luciana Lopes, Clifton Park, NY (US); Charles Robert Flynn, Chandler, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: Arizona Board of Regents, A body Corporate, of the State of Arizona, acting for and on behalf of; Arizona State University

(21) Appl. No.: 11/972,469

(22) Filed: Jan. 10, 2008

Related U.S. Application Data
(60) Provisional application No. 60/880,137, filed on Jan. 10, 2007.
(60) Provisional application No. 60/949,971, filed on Jul. 16, 2007.

(51) Int. Cl. A61K 38/00; C07K 7/00; C12N 15/11; C12N 15/00; C12N 5/06; A61P 9/00

(52) U.S. Cl. 514/14; 530/326; 536/23.1; 435/320.1; 435/325

(57) ABSTRACT

The present invention provides polypeptide inhibitors of HSP27 kinase, compositions thereof, and methods for using such polypeptides and compositions for various therapeutic uses.

### CROSS REFERENCE

This application claims priority to U.S. provisional patent application Ser. Nos. 60/880,137 filed Jan. 10, 2007 and 60/949,971 filed Jul. 16, 2007, both of which are incorporated by reference herein in their entirety.

### STATEMENT OF GOVERNMENT FUNDING

The U.S. Government through the National Institute of Health provided financial assistance for this project under NIH/NHLBI Grant Number R01 HL58027. Therefore, the United States Government has certain rights to this invention.

### FIELD OF THE INVENTION

The invention is in the fields of cell and molecular biology, polypeptides, drug discovery, and therapeutic methods of use.

### SUMMARY OF THE INVENTION

In a first aspect, the present invention provides a polypeptide comprising or consisting of a sequence according to general formula I:

| | |
|---|---|
| Z1-X1-LNRQLGVAA-Z2 | (SEQ ID NO:1) |

wherein Z1 and Z2 are independently absent or are transduction domains; and

X1 is selected from the group consisting of KA, KKA, and KKKA (SEQ ID NO: 46), or is absent, with the proviso that if X1 is KKKA (SEQ ID NO: 46), then at least one of Z1 and Z2 is a transduction domain, and wherein when X1 is absent, then Z1 is a transduction domain ending in KA.

In a preferred embodiment, at least one of Z1 and Z2 is a transduction domain.

In another aspect, the present invention provides compositions, comprising one or more polypeptides of the present invention and a pharmaceutically acceptable carrier.

In another aspect, the present invention provides isolated nucleic acid sequences encoding a polypeptide of the present invention. In further aspects, the present invention provides recombinant expression vectors comprising the nucleic acid sequences of the present invention, and host cells transfected with the recombinant expression vectors of the present invention.

In another aspect, the invention provides biomedical devices, wherein the biomedical devices comprise one or more polypeptides of the present invention disposed on or in the biomedical device. In various embodiments, such biomedical devices include stents, grafts, shunts, stent grafts, angioplasty devices, balloon catheters, fistulas, wound dressings, and any implantable drug delivery device.

In a further aspect, the present invention provides methods for one or more of the following therapeutic uses:

(a) reducing smooth muscle cell proliferation and/or migration; (b) promoting smooth muscle relaxation; (c) increasing the contractile rate in heart muscle; (d) increasing the rate of heart muscle relaxation; (e) promoting wound healing; (f) treating and/or reducing fibrotic disorders and/or keloids; (g) reducing scar formation; (h) disrupting focal adhesions; (i) regulating actin polymerization; and (j) treating or reducing incidence of one or more of intimal hyperplasia, stenosis, restenosis, atherosclerosis, smooth muscle cell tumors and metastasis, smooth muscle spasm, angina, Prinzmetal's angina, ischemia, stroke, bradycardia, hypertension, cardiac hypertrophy, renal failure, stroke, pulmonary hypertension, asthma, toxemia of pregnancy, pre-term labor, pre-eclampsia/eclampsia, Raynaud's disease or phenomenon, hemolytic-uremia, non-occlusive mesenteric ischemia, anal fissure, achalasia, impotence, migraine, ischemic muscle injury associated with smooth muscle spasm, vasculopathy, bradyarrythmia, bradycardia, congestive heart failure, stunned myocardium, pulmonary hypertension, diastolic dysfunction, gliosis; chronic obstructive pulmonary disease, osteopenia, endothelial dysfunction, inflammation, rheumatoid arthritis, degenerative arthritis, sepsis, endotoxemic shock, psoriasis, radiation enteritis, scleroderma, cirrhosis, interstitial fibrosis, Chrohn's disease, appendicitis, gastritis, laryngitis, meningitis, pancreatitis, otitsis, and reperfusion injury; wherein the method comprises administering to a subject in need thereof an effective amount to carry out the one or more therapeutic uses of one or more polypeptides or compositions according to the present invention, or functional equivalents thereof.

### BRIEF DESCRIPTION OF THE FIGURES

FIG. 1: BIP inhibits in vitro phosphorylation of HSP27 with MAPKAP kinase II. Immunoblot probed with antibodies against HSP27 and phospho-HSP27 to determine the ratio of P-P27 to P27 and the percent phosphorylation with respect to the phosphorylation obtained in the absence of inhibitor.

FIG. 2: Data graph showing that HSP27 kinase inhibitor peptide (BIP, 10 μM) inhibits TGF-β1 induced expression of CTGF and collagen and reduces the phosphorylation of HSP27 in human keloid fibroblasts.

FIG. 3: Data graph showing that HSP27 kinase inhibitor peptide (BIP, 60 μM) inhibits TGF-β1 induced expression of CTGF and collagen in human keloid fibroblasts.

FIG. 4: Data graph showing that HSP27 kinase inhibitor peptide (BIP, 60 μM) inhibits TGF-β1 induced phosphorylation of HSP27 in human keloid fibroblasts.

FIG. 5 Data graph showing BIP enhancement of sodium nitroprusside induced relaxation of saphenous vein.

FIG. 6 Data graph showing that BIP or SB treatment inhibits the thickening of the intimal layer in saphenous vein.

FIG. 7 (A) Immunoblot, and (B-C) data graphs showing that BIP or SB treatment inhibit CTGF expression and phosphorylation of HSP27 in saphenous vein in an organ culture model.

FIG. 8 (A-B) Immunoblots, and (C-D)O data graphs showing BIP reduces arsenic (ARS) or lysophosphatidic acid (LPA) induced phosphorylation of HSP27 in rat aortic smooth muscle cells (A7r5).

FIG. 9 (A) Immunoblot, and (B) data graph showing that BIP reduces arsenic (ARS) or lysophosphatidic acid (LPA) induced expression of CTGF in rat aortic smooth muscle cells (A7r5).

FIG. 10 Data graph showing that BIP inhibits migration of rat aortic smooth muscle (A7R5) cells.

### DETAILED DESCRIPTION OF THE INVENTION

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.)

The single letter designation for amino acids is used predominately herein. As is well known by one of skill in the art, such single letter designations are as follows:

A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is gluatamine; R is arginine; S is serine; T is threonine; V is valine; W is tryptophan; and Y is tyrosine.

As used herein, the singular forms “a”, “an” and “the” include plural referents unless the context clearly dictates otherwise. For example, reference to a “polypeptide” means one or more polypeptides.

In a first aspect, the present invention provides a polypeptide comprising or consisting of a sequence according to general formula I:

| | |
|---|---|
| Z1-X1-LNRQLGVAA-Z2 | (SEQ ID NO:1) |

wherein Z1 and Z2 are independently absent or are transduction domains; and

X1 is selected from the group consisting of KA, KKA, and KKKA (SEQ ID NO: 46), or is absent, with the proviso that if X1 is KKKA (SEQ ID NO: 46), then at least one of Z1 and Z2 is a transduction domain, and wherein when X1 is absent, then Z1 is a transduction domain ending in KA.

In a preferred embodiment, at least one of Z1 and Z2 are a transduction domain.

The polypeptides of the present invention are useful, for example, as HSP27 kinase inhibitors, which can be used as therapeutic agents for a variety of disorders, as disclosed in more detail below.

The term “polypeptide” is used in its broadest sense to refer to a sequence of subunit amino acids, amino acid analogs, or peptidomimetics. The subunits are linked by peptide bonds, except where noted. The polypeptides described herein may be chemically synthesized or recombinantly expressed.

Preferably, the polypeptides of the present invention are chemically synthesized. Synthetic polypeptides, prepared using the well known techniques of solid phase, liquid phase, or peptide condensation techniques, or any combination thereof, can include natural and unnatural amino acids. Amino acids used for peptide synthesis may be standard Boc (Nα-amino protected Nα-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile Nα-amino protected 9-fluorenylmethoxycarbonyl (Fmoc) amino acids first described by Carpino and Han (1972, J. Org. Chem. 37:3403-3409). Both Fmoc and Boc Nα-amino protected amino acids can be obtained from Sigma, Cambridge Research Biochemical, or other chemical companies familiar to those skilled in the art. In addition, the polypeptides can be synthesized with other Nα-protecting groups that are familiar to those skilled in this art.

Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, and norleucine for leucine or isoleucine.

In addition, the polypeptides can have peptidomimetic bonds, such as ester bonds, to prepare peptides with novel properties. For example, a peptide may be generated that incorporates a reduced peptide bond, i.e., R₁-CH₂-NH-R₂, (SEQ ID NO: 2) where R₁ and R₂ are amino acid residues or sequences. A reduced peptide bond may be introduced as a dipeptide subunit. Such a polypeptide would be resistant to protease activity, and would possess an extended half-live in vivo.

In a preferred embodiment, at least one of Z1 and Z2 is a transduction domain. As used herein, the term “transduction domain” means one or more polypeptide or any other molecule that can carry the active domain across cell membranes. These domains can be linked to other polypeptides to direct movement of the linked polypeptide across cell membranes. In some cases the transducing molecules do not need to be covalently linked to the active polypeptide. In a preferred embodiment, the transduction domain is linked to the rest of the polypeptide via peptide bonding. (See, for example, Cell 55: 1179-1188, 1988; Cell 55: 1189-1193, 1988; Proc Natl Acad Sci USA 91: 664-668, 1994; Science 285: 1569-1572, 1999; J Biol Chem 276: 3254-3261, 2001; and Cancer Res 61: 474-477, 2001) In a further embodiment, both X1 and X3 are transduction domains. In a further preferred embodiment, the transduction domain(s) is/are selected from the group consisting of (R)₄₉ (SEQ ID NO: 3); GRKKRRQRRRPPQ (SEQ ID NO: 4); RQRRKKRG (SEQ ID NO: 5); GRKKRRQR (SEQ ID NO: 6); AYARAAARQARA (SEQ ID NO: 7); DAATATRGRSAASRPTERPRAPARSASRPRRPVE (SEQ ID NO: 8); GWTLNSAGYLLGLINLKALAALAKKIL (SEQ ID NO: 9); PLSSIFSRIGDP (SEQ ID NO: 10); AAVALLPAVLLALLAP (SEQ ID NO: 11); AAVLLPVLLAAP (SEQ ID NO: 12); VTVLALGALAGVGVG (SEQ ID NO: 13); GALFLGWLGAAGSTMGAWSQP (SEQ ID NO: 14); GWTLNSAGYLLGLINLKALAALAKKIL (SEQ ID NO: 15); KLALKLALKALKAALKLA (SEQ ID NO: 16); KETWWETWWTEWSQPKKKRKV (SEQ ID NO: 17); KAFAKLAARLYRKA (SEQ ID NO: 18); KAFAKLAARLYRAA (SEQ ID NO: 19); AAFAKLAARLYRKA (SEQ ID NO: 20); KAFAALAARLYRKA (SEQ ID NO: 21); KAFAKLAARLYRKAGC (SEQ ID NO: 22); KAFAKLAARLYRAAGC (SEQ ID NO: 23); AAFAKLAARLYRKAGC (SEQ ID NO: 24); KAFAALAARLYRKAGC (SEQ ID NO: 25); KAFAKLAAQLYRKAGC (SEQ ID NO: 26), AGGGGYGRKKRRQRRR (SEQ ID NO: 27), and YARAAARQARA (SEQ ID NO: 28), YGRKKRRQRRR (SEQ ID NO: 29), WLRRIKAWLRRIKA (SEQ ID NO: 30); and WLRRIKAWLRRIKAWLRRIKA (SEQ ID NO: 31).

Further exemplary polypeptides according to the invention include, but are not limited to any of those listed above, wherein one or both of Z1 and Z2 are selected from the group consisting of WLRRIKAWLRRIKA (SEQ ID NO: 30); WLRRIKAWLRRIKAWLRRIKA (SEQ ID NO: 31); YGRKKRRQRRR (SEQ ID NO: 29); YARAAARQARA (SEQ ID NO: 28); RQRRKKRG (SEQ ID NO: 5); and GRKKRRQR (SEQ ID NO: 6).

In various further embodiments, exemplary polypeptides according to the invention include, but are not limited to those comprising or consisting of:

| | |
|---|---|
| YARAAARQARAKALNRQLGVAA; | (SEQ ID NO:32) |
| YGRKKRRQRRRKALNRQLGVAA; | (SEQ ID NO:33) |
| RQRRKKRGKALNRQLGVAA; | (SEQ ID NO:34) |
| GRKKRRQRKALNRQLGVAA; | (SEQ ID NO:35) |
| WLRRIKAWLRRIKAKALNRQLGVAA; | (SEQ ID NO:36) |
| WLRRIKAWLRRIKAWLRRIKAKALNRQLGVAA; | (SEQ ID NO:37) |
| YARAAARQARAKKKALNRQLGVAA; | (SEQ ID NO:38) |
| YGRKKRRQRRRKKKALNRQLGVAA; | (SEQ ID NO:39) |
| RQRRKKRGKKKALNRQLGVAA; | (SEQ ID NO:40) |
| GRKKRRQRKKKALNRQLGVAA; | (SEQ ID NO:41) |
| WLRRIKAWLRRIKAKKKALNRQLGVAA; | (SEQ ID NO:42) |
| and | |
| WLRRIKAWLRRIKAWLRRIKAKKKALNRQLGVAA. | (SEQ ID NO:43) |

In another aspect, the present invention provides compositions, comprising one or more of the polypeptides disclosed herein, and a pharmaceutically acceptable carrier. Such pharmaceutical compositions are especially useful for carrying out the methods of the invention described below. For administration, the polypeptides are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, dextran sulfate, heparin-containing gel or non-gel compositions or coatings, and/or polyvinyl alcohol, and tableted or encapsulated for conventional administration. Alternatively, the polypeptides of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art. The polypeptides may be linked to other compounds to promote an increased half-life in vivo, such as polyethylene glycol. Such linkage can be covalent or non-covalent as is understood by those of skill in the art.

The polypeptides may be made up in a solid form (including granules, powders or suppositories) or in a liquid form (e.g., solutions, suspensions, or emulsions). The polypeptides of the invention may be applied in a variety of solutions. Suitable solutions for use in accordance with the invention are sterile, dissolve sufficient amounts of the polypeptides, and are not harmful for the proposed application.

In another aspect, the present invention provides an isolated nucleic acid encoding a polypeptide of the present invention. Appropriate nucleic acids according to this aspect of the invention will be apparent to one of skill in the art based on the disclosure provided herein and the general level of skill in the art.

In another aspect, the present invention provides an expression vector comprising DNA control sequences operably linked to the isolated nucleic acids of the present invention, as disclosed above. “Control sequences” operably linked to the nucleic acids of the invention are nucleic acid sequences capable of effecting the expression of the nucleic acids of the invention. The control sequences need not be contiguous with the nucleic acids, so long as they function to direct the expression thereof. Thus, for example, intervening untranslated yet transcribed sequences can be present between a promoter sequence and the nucleic acid and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors.

In a further aspect, the present invention provides genetically engineered host cells comprising the expression vectors of the invention. Such host cells can be prokaryotic cells or eukaryotic cells, and can be either transiently or stably transfected, or can be transduced with viral vectors.

In another aspect, the invention provides biomedical devices comprising one or more of the polypeptides of the present invention disposed on or in the biomedical device. As used herein, a “biomedical device” refers to a device to be implanted into a subject, for example, a human being, in order to bring about a desired result. Particularly preferred biomedical devices according to this aspect of the invention include, but are not limited to, stents (including but not limited to coronary stents), grafts (including but not limited to vascular grafts), shunts, stent grafts, fistulas, angioplasty devices, balloon catheters, venous catheters, implantable drug delivery devices, adhesion barriers (including but not limited to carboxymethylcellulose, hyaluronic acid, and PTFE sheets) to separate tissue, wound dressings such as films (e.g., polyurethane films), hydrocolloids (hydrophilic colloidal particles bound to polyurethane foam), hydrogels (cross-linked polymers containing about at least 60% water), other viscous liquids and hydrogel-like species (including but not limited to, those disclosed in US 20030190364), foams (hydrophilic or hydrophobic), calcium alginates (nonwoven composites of fibers from calcium alginate), cellophane, pluronics (ie: poly(ethylene glycol)-block-poly(propylene glycol), and biological polymers.

As used herein, the term “grafts” refers to both natural and prosthetic grafts and implants. In a preferred embodiment, the graft is a vascular graft.

As used herein, the term “stent” includes the stent itself, as well as any sleeve or other component that may be used to facilitate stent placement.

As used herein, “disposed on or in” means that the one or more polypeptides can be either directly or indirectly in contact with an outer surface, an inner surface, or embedded within the biomedical device. “Direct” contact refers to disposition of the polypeptides directly on or in the device, including but not limited to soaking a biomedical device in a solution containing the one or more polypeptides, spin coating or spraying a solution containing the one or more polypeptides onto the device, implanting any device that would deliver the polypeptide, and administering the polypeptide through a catheter directly on to the surface or into any organ.

“Indirect” contact means that the one or more polypeptides do not directly contact the biomedical device. For example, the one or more polypeptides may be disposed in a matrix, such as a gel matrix (such as a heparin coating) or a viscous fluid, which is disposed on the biomedical device. Such matrices can be prepared to, for example, modify the binding and release properties of the one or more polypeptides as required. In one non-limiting example, a heparin coating is disposed on the biomedical device (such as a poly(tetrafluoroethylene) (PTFE) vascular device or sheet) and the one or more polypeptides are disposed on or in a heparin coating; in this example, the one or more polypeptides can be delivered to a subject in need thereof in a controlled manner. In one non-limiting example, the release of the one or more polypeptides from interstitial surfaces of poly(tetrafluoroethylene) (PTFE) vascular devices or sheets can be controlled by first adsorbing or bonding heparin to the surface and/or interstices of the PTFE device followed by adsorption of polypeptide. Alternating layers of heparin and the polypeptide can also be used to increase the polypeptide dose and/or time of release. Under physiological conditions within the body, the kinetics of the association and dissociation of polypeptides disclosed herein to and from heparin will lead to a delayed release profile as compared to release of the polypeptide from a bare PTFE device. In addition, the release profile can be further altered through changes in local temperature, pH or ionic strength. Such controlled release is of great value for use in the various therapeutic treatments for which the biomedical devices can be used, as discussed below.

Heparin coatings on various medical devices are known in the art. Applications in humans include central venous catheters, coronary stents, ventricular assist devices, extracorporeal blood circuits, blood sampling devices, and vascular grafts. Such coatings can be in a gel or non-gel form. As used herein “heparin coating” includes heparin adsorbed to the surface, heparin bonded to the surface, and heparin imbedded in the PTFE polymer surface. An example of a method for bonding the heparin would be to use ammonia plasma to treat, for example, a PTFE surface and reacting the resultant amines with oxidized heparin. Layer-by-layer buildup of the heparin and one or more polypeptides could then be used to increase polypeptide on the surface and expand the delivery time. Gel forms of the heparin coating can include, but are not limited to, any hydrogel containing heparin either covalently or physically bound to the gel. The heparin coating is disposed on the biomedical device, which includes direct contact with an outer surface or an inner surface of the biomedical device, or embedded within the biomedical device. “Direct” contact refers to disposition directly on or in the device, including but not limited to soaking a biomedical device in a heparin coating solution (wherein the polypeptides may be added as part of the heparin coating solution, or may be subsequently disposed on or in the heparin coating after it is contacted with the device), spin coating or spraying a heparin coating solution onto the device (wherein the polypeptides may be added as part of the heparin coating solution, or may be subsequently disposed on or in the heparin coating after it is contacted with the device), and administering the heparin coating solution containing the polypeptides through a catheter directly on to the surface or into any organ. The physical characteristics and specific composition of the heparin layer can be any that provides the desired release profile of the one or more polypeptides. See, for example, Seal and Panitch, Biomacromolecules 2003(4):1572-1582 (2003); US20030190364, incorporated by reference herein in its entirety; and Carmeda BioActive Surface (CBAS™) the product of Carmeda AB in Stockholm, Sweden. “Indirect” contact means that the heparin coating is not directly in contact with the device such as, for example, when an intervening coating is placed between the device surface and the heparin coating. In one non-limiting example, the one or more polypeptides could be initially adsorbed (directly or indirectly), and then adsorbing a heparin coating; this can optionally be followed by subsequent polypeptide layers, heparin layers, or combinations thereof, as desired. As will be understood by those of skill in the art, any sulfated polysaccharide or negatively charged polymer can be used in like manner to heparin as described above, to provide desired release characteristics.

In a further aspect, the present invention provides methods for one or more of the following therapeutic uses

(a) reducing smooth muscle cell proliferation and/or migration; (b) promoting smooth muscle relaxation; (c) increasing the contractile rate in heart muscle; (d) increasing the rate of heart muscle relaxation; (e) promoting wound healing; (f) treating and/or reducing fibrotic disorders and/or keloids; (g) reducing scar formation; (h) disrupting focal adhesions; (i) regulating actin polymerization; and (j) treating or reducing incidence of one or more of intimal hyperplasia, stenosis, restenosis, atherosclerosis, smooth muscle cell tumors and metastasis, smooth muscle spasm, angina, Prinzmetal's angina (coronary vasospasm), ischemia, stroke, bradycardia, hypertension, cardiac hypertrophy and other end-organ damage associated with hypertension (including but not limited to renal failure and stroke), pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia, anal fissure, achalasia, impotence, migraine, ischemic muscle injury associated with smooth muscle spasm, vasculopathy, such as transplant vasculopathy; bradyarrythmia, bradycardia, congestive heart failure, stunned myocardium, pulmonary hypertension, diastolic dysfunction, gliosis (proliferation of astrocytes, and may include deposition of extracellular matrix, including but not limited to such proliferation and ECM deposition in damaged areas of the central nervous system; chronic obstructive pulmonary disease (COPD) (ie, respiratory tract diseases characterized by airflow obstruction or limitation; includes but is not limited to chronic bronchitis and emphysema), bone resorption (osteopenia) associated with aging or immobilization (which leads to bone fractures); limiting endothelial dysfunction, inflammation, rheumatoid arthritis, degenerative arthritis, sepsis, endotoxemic shock, psoriasis, radiation enteritis, scleroderma, cirrhosis, interstitial fibrosis, Chrohn's disease, appendicitis, gastritis, laryngitis, meningitis, pancreatitis, otitsis, and reperfusion injury;

wherein the method comprises administering to a subject in need thereof an effective amount to carry out the one or more therapeutic uses of one or more polypeptides or compositions according to the present invention, or functional equivalents thereof.

While not being bound by any specific mechanism, the inventors believe that the polypeptides of the present invention provide their therapeutic effect as a result of inhibiting HSP27 phosphorylation by HSP27 kinase (MAPKAP2), although alternative mechanisms, including but note limited to inhibition of HSP27 phosphorylation by MAPKAP3, and MAPKAP5 are also encompassed by the present invention.

HSP27 is related to the other small heat shock proteins and has been shown to alter cytoskeletal dynamics. Transfection of cells with dominant active phosphorylated mutants of HSP27 leads to actin stress fiber formation (Mol Cell Biol. 15: 505-516, 1995; J Biol. Chem. 268, 24210-24214, 1993). This is associated with the myofibroblast or scar phenotype and promotes fibrosis. Furthermore, increases in the phosphorylation of HSP27 are associated with smooth muscle cell migration (J Biol. Chem. 274, 24211-24219, 1999). Both cellular migration and myofibroblast formation are associated with wound healing and when overly exuberant can promote scar formation (Journal of Surgical Research 102, 77-84, 2002). In addition, smooth muscle actin expression and myofibroblast differentiation by transforming growth factor beta are dependent on MAPKAP2 (J. Cell Biochem. 2006). Taken together, this suggests that inhibiting MAKAP2 would inhibit fibrosis.

Increases in the phosphorylation of HSP27 are also associated with smooth muscle contraction (Am J Physiol. Heart Circ Physiol. 278, H1899-1907, 2000). Thus, inhibiting the phosphorylation of HSP27 would prevent contraction associated with overly exuberant smooth muscle contraction. MAPKAP2 has also been implicated in inflammation and cancer (Nat. Rev. Mol. Cell. Biol. 7, 120-130, 2006; Oncogene 25, 2987-2998, 2006). Furthermore, since MAPKAP2 is downstream of p38 MAP kinase, any therapeutic uses for which p38 MAPK inhibitors are useful are within the scope of the present invention as well.

In a preferred embodiment, the individual is a mammal; in a more preferred embodiment, the individual is a human.

As used herein, “treat” or “treating” means accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

As used herein, the term “reduce” or “reducing” means to limit occurrence of the disorder in individuals at risk of developing the disorder.

As used herein, “administering” includes in vivo administration, as well as administration directly to tissue ex vivo, such as vein grafts.

Intimal hyperplasia is a complex process that leads to graft failure, and is the most common cause of failure of arterial bypass grafts. While incompletely understood, intimal hyperplasia is mediated by a sequence of events that include endothelial cell injury and subsequent vascular smooth muscle proliferation and migration from the media to the intima. This process is associated with a phenotypic modulation of the smooth muscle cells from a contractile to a synthetic phenotype. The “synthetic” smooth muscle cells secrete extracellular matrix proteins, which leads to pathologic narrowing of the vessel lumen leading to graft stenoses and ultimately graft failure. Such endothelial cell injury and subsequent smooth muscle cell proliferation and migration into the intima also characterize restenosis, most commonly after angioplasty to clear an obstructed blood vessel.

In some embodiments of the methods of the invention, such as those relating to reducing occurrence of smooth muscle cell proliferation and/or migration, or promoting smooth muscle relaxation, the administering may be direct, by contacting a blood vessel in a subject being treated with one or more polypeptides of the invention. For example, a liquid preparation of one or more polypeptides according to the invention can be forced through a porous catheter, or otherwise injected through a catheter to the injured site, or a gel or viscous liquid containing the one or more polypeptides according to the invention can be spread on the injured site. In these embodiment of direct delivery, it is most preferred that the one or more polypeptides according to the invention be delivered into smooth muscle cells at the site of injury or intervention. This can be accomplished, for example, by delivering the recombinant expression vectors (most preferably a viral vector, such as an adenoviral vector) of the invention to the site. More preferably, delivery into smooth muscle cells is accomplished by using the one or more polypeptides according to the invention that include at least one transduction domain to facilitate entry into the smooth muscle cells.

In various other preferred embodiments of this methods of the invention, particularly those that involve reducing occurrence of smooth muscle cell proliferation and/or migration, the method is performed on a subject who has undergone, is undergoing, or will undergo a procedure selected from the group consisting of angioplasty, vascular stent placement, endarterectomy, atherectomy, bypass surgery (such as coronary artery bypass surgery; peripheral vascular bypass surgeries), vascular grafting, organ transplant, prosthetic device implanting, microvascular reconstructions, plastic surgical flap construction, and catheter emplacement.

In another embodiment, the methods comprise treating or reducing occurrence of one or more disorder selected from the group consisting of intimal or neointimal hyperplasia, stenosis, restenosis, and atherosclerosis, comprising contacting a subject in need thereof with an amount effective to treat or reduce intimal or neointimal hyperplasia, stenosis, restenosis, and/or atherosclerosis of one or more polypeptides according to the invention.

In a further embodiment of this aspect of the invention, the method is used to treat tumors and/or metastasis, including but not limited to smooth muscle tumors. In one embodiment, the tumor is a leiomyosarcoma, which is defined as a malignant neoplasm that arises from muscle. Since leiomyosarcomas can arise from the walls of both small and large blood vessels, they can occur anywhere in the body, but peritoneal, uterine, and gastro-intestinal (particularly esophageal) leiomyosarcomas are more common. Alternatively, the smooth muscle tumor can be a leiomyoma, a non-malignant smooth muscle neoplasm. In a further embodiment, the method can be combined with other treatments for smooth muscle cell tumors and/or metastasis, such as chemotherapy, radiation therapy, and surgery to remove the tumor. While not being limited by any specific mechanism, the inventors believe that administration of the polypeptides of the invention can be used to treat tumors and/or metastasis by any or all of the following mechanisms: preventing drug resistance to anticancer drugs or promoting susceptibility to anti cancer drugs as an MK2 (MAPKAPII) kinase inhibitor, promoting apoptosis of cancer cells, decreasing cell invasion through decreased matrix metalloproteinase expression and decreased migration of cancer cells, and through suppressing viral oncogenesis.

In a further embodiment, the methods of the invention are used for treating or reducing occurrence of smooth muscle spasm, comprising contacting a subject or graft in need thereof with an amount effective to reduce smooth muscle spasm of one or more polypeptides according to the invention.

Smooth muscles are found in the walls of blood vessels, airways, the gastrointestinal tract, and the genitourinary tract. Pathologic tonic contraction of smooth muscle constitutes spasm. Many pathological conditions are associated with spasm of vascular smooth muscle (“vasospasm”), the smooth muscle that lines blood vessels. This can cause symptoms such as angina and ischemia (if a heart artery is involved), or stroke as in the case of subarachnoid hemorrhage induced vasospasm if a brain vessel is involved. Hypertension (high blood pressure) is caused by excessive vasoconstriction, as well as thickening, of the vessel wall, particularly in the smaller vessels of the circulation.

Thus, in a further embodiment of the methods of the invention, the muscle cell spasm comprises a vasospasm, and the methods of the invention are used to treat or reduce occurrence of vasospasm. Preferred embodiments of the method include, but are not limited to, methods to treat or inhibit angina, coronary vasospasm, Prinzmetal's angina (episodic focal spasm of an epicardial coronary artery), ischemia, stroke, bradycardia, and hypertension.

In another embodiment of the methods of the invention, occurrence of smooth muscle spasm is reduced by treatment of a graft, such as a vein or arterial graft, with the one or more polypeptides according to the invention. One of the ideal conduits for peripheral vascular and coronary reconstruction is the greater saphenous vein. However, the surgical manipulation during harvest of the conduit often leads to vasospasm. The exact etiology of vasospasm is complex and most likely multifactorial. Most investigations have suggested that vasospasm is either due to enhanced constriction or impaired relaxation of the vascular smooth muscle in the media of the vein. Numerous vasoconstricting agents such as endothelin-1 and thromboxane are increased during surgery and result in vascular smooth muscle contraction. Other vasoconstrictors such as norepinephrine, 5-hydroxytryptamine, acetylcholine, histamine, angiotensin II, and phenylephrine have been implicated in vein graft spasm. Papaverine is a smooth muscle vasodilator that has been used. In circumstances where spasm occurs even in the presence of papaverine, surgeons use intraluminal mechanical distension to break the spasm. This leads to injury to the vein graft wall and subsequent intimal hyperplasia. Intimal hyperplasia is the leading cause of graft failure.

Thus, in this embodiment, the graft can be contacted with the one or more polypeptides according to the invention, during harvest from the graft donor, subsequent to harvest (before implantation), and/or during implantation into the graft recipient (ie: ex vitro or in vivo). This can be accomplished, for example, by delivering the recombinant expression vectors (most preferably a viral vector, such as an adenoviral vector) of the invention to the site, and transfecting the smooth muscle cells. More preferably, delivery into smooth muscle is accomplished by using the one or more polypeptides according to the invention that include at least one transduction domain to facilitate entry into the smooth muscle cells. During graft implantation, it is preferred that the subject receiving the graft be treated systemically with heparin, as heparin has been shown to bind to protein transduction domains and prevent them from transducing into cells. This approach will lead to localized protein transduction of the graft alone, and not into peripheral tissues. The methods of this embodiment of the invention reduce occurrence of vein graft spasm during harvest and/or implantation of the graft, and thus improve both short and long term graft success.

In various other embodiments of the methods of the invention, the muscle cell spasm is associated with a disorder including, but not limited to pulmonary (lung) hypertension, asthma (bronchospasm), toxemia of pregnancy, pre-term labor, pre-eclampsia/eclampsia, Raynaud's disease or phenomenon, hemolytic-uremia, non-occlusive mesenteric ischemia (ischemia of the intestines that is caused by inadequate blood flow to the intestines), anal fissure (which is caused by persistent spasm of the internal anal sphincter), achalasia (which is caused by persistent spasm of the lower esophageal sphincter), impotence (which is caused by a lack of relaxation of the vessels in the penis, erection requires vasodilation of the corpra cavernosal (penile) blood vessels), migraine (which is caused by spasm of the intracranial blood vessels), ischemic muscle injury associated with smooth muscle spasm, and vasculopathy, such as transplant vasculopathy (a reaction in the transplanted vessels which is similar to atherosclerosis, it involves constrictive remodeling and ultimately obliteration of the transplanted blood vessels, this is the leading cause of heart transplant failure).

In other embodiments, the methods of the invention are used for one or more of promoting wound healing, reducing scar formation, treating and/or reducing fibrotic disorders and treating and/or reducing keloids. In these embodiments, an “individual in need thereof” is an individual that has suffered or will suffer (for example, via a surgical procedure) a wound that may result in scar formation, or has resulted in scar formation. As used herein, the term “wound” refers broadly to injuries to the skin and subcutaneous tissue. Such wounds include, but are not limited to lacerations; burns; punctures; pressure sores; bed sores; canker sores; trauma, bites; fistulas; ulcers; lesions caused by infections; periodontal wounds; endodontic wounds; burning mouth syndrome; laparotomy wounds; surgical wounds; incisional wounds; contractures after burns; tissue fibrosis, including but not limited to idiopathic pulmonary fibrosis, hepatic fibrosis, renal fibrosis, retroperitoneal fibrosis, and cystic fibrosis, but excluding blood vessel fibrosis or heart tissue fibrosis; and wounds resulting from cosmetic surgical procedures. In these embodiments, it is preferred that the one or more polypeptides or compositions are disposed on or in a wound dressing or other topical administration. Such wound dressings can be any used in the art, including but not limited to films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), cellophane, and biological polymers such as those described in US patent application publication number 20030190364, published Oct. 9, 2003.

As used herein, the phrase “reducing scar formation” means any decrease in scar formation that provides a therapeutic or cosmetic benefit to the patient. Such a therapeutic or cosmetic benefit can be achieved, for example, by decreasing the size and/or depth of a scar relative to scar formation in the absence of treatment with the methods of the invention, or by reducing the size of an existing scar. As used herein, such scars include scars of all types, including but not limited to keloids; hypertrophic scars; and adhesion formation between organ surfaces, including but not limited to those occurring as a result of surgery.

The methods of these embodiments are clinically useful for treating all types of wounds to reduce scar formation, both for reducing initial scar formation, and for therapeutic treatment of existing scars (i.e.: cutting out the scar after its formation, treating it with the compounds of the invention, and letting the scar heal more slowly). In a preferred embodiment, individuals in need of treatment or limiting of scarring (such as keloids or hypertrophic scarring) are highly pigmented individuals, including but not limited to individuals of Asian or African descent, that are susceptible to keloids, and thus can benefit from the methods of the invention for prophylactic therapy to limit development of keloids, as well as for treating keloids. In various other preferred embodiments, individuals in need of therapy for treating or limiting fibrotic disorders are those suffering from or at risk of one or more fibrotic disorders associated with TGFβ-induced CTGF expression, including but not limited to tissue fibrosis (including but not limited to idiopathic pulmonary fibrosis, hepatic fibrosis, renal fibrosis, retroperitoneal fibrosis, cystic fibrosis, blood vessel fibrosis, CNS fibrosis, and heart tissue fibrosis); diabetic nephropathy, glomerulosclerosis, and IgA nephropathy (causes of kidney failure and the need for dialysis and retransplant); diabetic retinopathy and macular degeneration (fibrotic diseases of the eye and leading causes of blindness); cirrhosis and biliary atresia (leading causes of liver fibrosis and failure); congestive heart failure; lung fibrosis; scleroderma; abdominal adhesions; and interstitial fibrosis.

In various other preferred embodiments of all of the embodiments disclosed herein, individuals in need of therapy for treating and/or limiting fibrotic disorders and/or keloids are those with elevated levels of one or more of the following biomarkers:

TGFβ1 expression;

Collagen I;

CTGF expression; and

alpha smooth muscle actin.

Elevated levels of such biomarkers can be detected using standard techniques, including but not limited to immunological techniques (ELISA, immunocytochemistry, etc.) using commercially available antibodies against the one or more biomarkers.

As disclosed below, the polypeptides of the invention inhibit TGFβ1-induced CTGF and collagen expression in human keloid fibroblasts, which are elevated in fibrotic conditions, indicating that individuals with elevated levels of one or more of these biomarkers can especially benefit from the methods of the present invention. As used herein, an “elevated” level of the one or more biomarkers means any increase above normal for that individual or similarly situated individuals in a relevant target tissue. Such target tissues are those affected by fibrotic conditions, including but not limited to blood, wound exudate, and biopsies taken from tissues affected by fibrosis including but not limited to those disclosed above (skin, kidney, lung, liver, peritoneum, blood vessel, heart, retina, etc.) In various further embodiments, an individual in need thereof is one that has a level of one or more of the recited biomarkers 5%, 10%, 15%, 20%, 25%, 50%, 75%, 100%, or more above normal levels. Determining the level of the one or more biomarkers can be done using standard techniques in the art for measuring protein and/or gene expression, including but not limited to those disclosed below.

A “normal” level of these one or more biomarkers may be established by any suitable means, including but not limited to determining a normal level in that individual or similarly situated individuals in the absence of fibrotic conditions and/or keloids, or any other suitable means to establish a standard for reference.

In another embodiment, the methods comprise treating reperfusion injury and/or reducing the incidence of reperfusion injury. As used herein, “reperfusion injury” refers to damage to tissue caused when its blood supply returns to the tissue after a period of ischemia, in which the restoration results in inflammation, apoptosis, and oxidative damage to the tissue. Examples of reperfusion injury treated or reduced by the methods of the invention include reperfusion injury associated with stroke, myocardial infarction, peripheral ischemia, and trauma.

Preferred routes of delivery for these various indications of the different embodiments of the methods of the invention vary. Topical administration is preferred for methods involving treatment or reducing the incidence of vein graft spasm, intimal hyperplasia, restenosis, prosthetic graft failure due to intimal hyperplasia, stent, stent graft failure due to intimal hyperplasia/constrictive remodeling, microvascular graft failure due to vasospasm, transplant vasculopathy, scarring, fibrosis, keloid formation, male and female sexual dysfunction, prevention of hydrocephalus caused by subarachnoid hemorrhage, and for promoting wound healing. As used herein, “topical administration” refers to delivering the polypeptide onto the surface of the organ.

Intrathecal administration, defined as delivering the polypeptide into the cerebrospinal fluid is the preferred route of delivery for treating or reducing incidence of stroke and subarachnoid hemorrhage induced vasospasm. Intraperitoneal administration, defined as delivering the polypeptide into the peritoneal cavity, is the preferred route of delivery for treating or reducing incidence of non-occlusive mesenteric ischemia. Oral administration is the preferred route of delivery for treating or reducing incidence of achalasia. Intravenous administration is the preferred route of delivery for treating or reducing incidence of hypertension and bradycardia. Administration via suppository is preferred for treating or reducing incidence of anal fissure. Aerosol delivery is preferred for treating or reducing incidence of asthma (ie: bronchospasm). Intrauterine administration is preferred for treating or reducing incidence of pre-term labor and pre-eclampsia/eclampsia.

In another embodiment of the methods of the invention, the methods are used to increase the contractile rate in heart muscle. Individuals that can benefit from such treatment include those who exhibit a reduced heart rate relative to either a normal heart rate for the individual, or relative to a “normal” heart rate for a similarly situated individual. As used herein, the phrase “increasing the contractile rate in heart muscle” means any increase in contractile rate that provides a therapeutic benefit to the patient. Such a therapeutic benefit can be achieved, for example, by increasing the contractile rate to make it closer to a normal contractile rate for the individual, a normal contractile rate for a similarly situated individual, or some other desired target contractile rate. In a preferred embodiment, the methods result in an increase of at least 5% in the contractile rate of the patient in need of such treatment. In further preferred embodiments, the methods of the invention result in an increase of at least 10%, 15%, 20%, 25%, 30%, 35%, 40%, 45%, and/or 50% in the contractile rate of the patient in need of such treatment. In a preferred embodiment, increasing the contractile rate in heart muscle is accomplished by increasing the heart muscle relaxation rate (ie: if the muscles relax faster, they beat faster). In a more preferred embodiment, the methods of the invention result in an increase of at least 5% in the heart muscle relaxation rate of the patient in need of such treatment. In further preferred embodiments, the methods of the invention result in an increase of at least 10%, 15%, 20%, 25%, 30%, 35%, 40%, 45%, and/or 50% in the heart muscle relaxation rate of the patient in need of such treatment.

In a further embodiment of the methods of the invention, the methods are performed to treat one or more cardiac disorders that can benefit from increasing the contractile rate in heart muscle. Such cardiac disorders include bradyarrythmias, bradycardias congestive heart failure, pulmonary hypertension, stunned myocardium, and diastolic dysfunction. As used herein, “bradyarrythmia” means an abnormal decrease of the rate of the heartbeat to less than 60 beats per minute, generally cased by a disturbance in the electrical impulses to the heart. A common cause of bradyarrythmias is coronary heart disease, which leads to the formation of atheromas that limit the flow of blood to the cardiac tissue, and thus the cardiac tissue becomes damaged. Bradyarrythmias due to coronary artery disease occur more frequently after myocardial infarction. Symptoms include, but are not limited to, loss of energy, weakness, syncope, and hypotension.

As used herein, “Congestive heart failure” means an inability of the heart to pump adequate supplies of blood throughout the body. Such heart failure can be due to a variety of conditions or disorders, including but not limited to hypertension, anemia, hyperthyroidism, heart valve defects including but not limited to aortic stenosis, aortic insufficiency, and tricuspid insufficiency; congenital heart defects including but not limited to coarctation of the aorta, septal defects, pulmonary stenosis, and tetralogy of Fallot; arrythmias, myocardial infarction, cardiomyopathy, pulmonary hypertension, and lung disease including but not limited to chronic bronchitis and emphysema. Symptoms of congestive heart failure include, but are not limited to, fatigue, breathing difficulty, pulmonary edema, and swelling of the ankles and legs.

As used herein, “Stunned myocardium” means heart muscle that is not functioning (pumping/beating) due to cardiac ischemia (lack of blood flow/oxygen to the vessels supplying the heat muscle).

As used herein, “Diastolic dysfunction” means an inability of the heart to fill with blood during diastole (the resting phase of heart contraction). This condition usually occurs in the setting of left ventricular hypertrophy. The heart muscle becomes enlarged and stiff such that it cannot fill adequately. Diastolic dysfunction can result in heart failure and inadequate heart function.

As used herein, “Pulmonary hypertension” means a disorder in which the blood pressure in the arteries supplying the lungs is abnormally high. Causes include, but are not limited to, inadequate supply of oxygen to the lungs, such as in chronic bronchitis and emphysema; pulmonary embolism, and intestinal pulmonary fibrosis. Symptoms and signs of pulmonary hypertension are often subtle and nonspecific. In the later stages, pulmonary hypertension leads to right heart failure that is associated with liver enlargement, enlargement of veins in the neck and generalized edema.

In a further embodiment of the methods of the invention, the methods are used for treating a heart muscle disorder comprising administering to an individual suffering from one or more of bradyarrythmia, bradycardia, congestive heart failure, stunned myocardium, pulmonary hypertension, and diastolic dysfunction, an amount effective to increase heart muscle contractile rate of one or more polypeptides according to the present invention.

Treating bradyarrythmia includes one or more of the following (a) improving the rate of the heartbeat to closer to normal levels for the individual, closer to a desired rate, or increasing to at least above 60 beats per minute; (b) reducing the occurrence of one or more of loss of energy, weakness, syncope, and hypotension in patients suffering from bradyarrythmia; (c) reducing worsening of one or more of loss of energy, weakness, syncope, and hypotension in patients suffering from bradyarrythmia and its symptoms; (d) reducing recurrence of bradyarrythmia in patients that previously suffered from bradyarrythmia; and (e) reducing recurrence of one or more of loss of energy, weakness, syncope, and hypotension in patients that previously suffered from bradyarrythmia.

Similarly, treating congestive heart failure includes one or more of the following (a) improving the heart's ability to pump adequate supplies of blood throughout the body to closer to normal levels for the individual, or closer to a desired pumping capacity; (b) reducing development of one or more of fatigue, breathing difficulty, pulmonary edema, and swelling of the ankles and legs in patients suffering from congestive heart failure; (c) reducing worsening of one or more of fatigue, breathing difficulty, pulmonary edema, and swelling of the ankles and legs in patients suffering from congestive heart failure and its symptoms; (d) reducing recurrence of congestive heart failure in patients that previously suffered from congestive heart failure; and (e) reducing recurrence of one or more of fatigue, breathing difficulty, pulmonary edema, and swelling of the ankles and legs in patients that previously suffered from congestive heart failure.

Treating stunned myocardium means one or more of (a) improving the ability of the heart muscle to pump by improving the oxygenation of the ischemic muscle, or by decreasing the need of the myocardial cells for oxygen and (b) reducing recurrence of stunned myocardium in patients that previously suffered from stunned myocardium.

Similarly, treating diastolic dysfunction includes one or more of (a) reducing occurrence of heart failure and/or inadequate heart function by allowing the heart to relax and fill more completely; (b) reducing recurrence of diastolic dysfunction in patients that previously suffered from diastolic dysfunction; and (c) reducing recurrence of heart failure and/or inadequate heart function in patients that previously suffered from diastolic dysfunction.

Treating pulmonary hypertension includes one or more of the following (a) decreasing blood pressure in the arteries supplying the lungs to closer to normal levels for the individual, or closer to a desired pressure; (b) reducing the occurrence of one or more of enlargement of veins in the neck, enlargement of the liver, and generalized edema in patients suffering from pulmonary hypertension; (c) reducing worsening of one or more of enlargement of veins in the neck, enlargement of the liver, and generalized edema in patients suffering from pulmonary hypertension and its symptoms; (d) reducing recurrence of pulmonary hypertension in patients that previously suffered from pulmonary hypertension; and (e) reducing recurrence of one or more of enlargement of veins in the neck, enlargement of the liver, and generalized edema in patients that previously suffered from pulmonary hypertension.

In a further aspect, the present invention provides methods for reducing occurrence of a heart muscle disorder comprising administering to an individual at risk of developing bradyarrythmia, bradycardia, congestive heart failure, stunned myocardium, pulmonary hypertension, and diastolic dysfunction an amount effective to increase heart muscle contractile rate of one or more polypeptides or compositions according to the present invention.

For example, methods to reduce occurrence of congestive heart failure involve administration of one or more polypeptides or compositions according to the present invention to a subject that suffers from one or more of hypertension, anemia, hyperthyroidism, heart valve defects including but not limited to aortic stenosis, aortic insufficiency, and tricuspid insufficiency; congenital heart defects including but not limited to coarctation of the aorta, septal defects, pulmonary stenosis, and tetralogy of Fallot; arrythmias, myocardial infarction, cardiomyopathy, pulmonary hypertension, and lung disease including but not limited to chronic bronchitis and emphysema.

Similarly, methods to reduce occurrence of bradyarrythmia involve administration of the one or more polypeptides or compositions according to the present invention to a subject that suffer from one or more of coronary heart disease and atheroma formation, or that previously had a myocardial infarction or conduction disorder.

Similarly, methods to reduce occurrence of pulmonary hypertension involve administration of the one or more polypeptides or compositions according to the present invention to a subject that suffers from one or more of chronic bronchitis, emphysema, pulmonary embolism, and intestinal pulmonary fibrosis.

Reducing occurrence of stunned myocardium involves administration of the one or more polypeptides or compositions according to the present invention to a subject that suffers from cardiac ischemia.

Reducing occurrence of or treating diastolic dysfunction involves administration of the one or more polypeptides or compositions according to the present invention to a subject that suffers from left ventricular hypertrophy

In other embodiments, the methods of the invention are used to promote the incidence of inducing neural regeneration for central nervous system injuries. As used herein, “neural regeneration” includes both regenerating a damaged neural connection, as well as promoting an increase in neural function (including but not limited to treatment of Alzheimer's and peripheral neuropathy); such neural regeneration can be in peripheral nervous system or the central nervous system. While not being limited by any specific mechanism of action, the inventors believe that administration of the peptides to a patient in need thereof prevents or limits activity of the protein rho, which is known to cause growth cone collapse; thus, minimizing rho activity enhances neurite outgrowth.

In other embodiments, the methods of the invention are used to treat or limit the incidence of gliosis (proliferation of astrocytes in damaged areas of the central nervous system). Astrocytes are the connective tissue cells of the CNS, and have functions including accumulating in areas with damaged neurons neurons. Gliosis occurs during any traumatic brain injury, insertion of neural electrodes and during spinal cord injury, as well as in various neurodegenerative disorders including but not limited to Korsakoff's syndrome and AIDS dementia complex. While not being limited by any specific mechanism of action, the inventors believe that administration of the peptides to a patient in need thereof prevents or limits the fibrotic response of astrocytes and possibly microglia to inhibit fibrosis.

In other embodiments, the methods of the invention are used to treat or limit the incidence of chronic obstructive pulmonary disease (COPD), which is a group of respiratory tract diseases characterized by airflow obstruction or limitation. COPD can be caused by a variety of factors, including but not limited to tobacco smoking (chronic smokers at risk), exposure to coal dust (coal minining industry workers particularly at risk), congenital defects (including but not limited to alpha 1-antitrypsin deficiency), or it may be idiopathic (no known cause). COPD includes, but is not limited to chronic bronchitis and emphysema. Symptoms characteristic of COPD (for which the methods of the invention can be used to treat or reduce incidence of) include, but are not limited to recurrent respiratory infections, severe cough, constant wheezing, shortness of breath with minimal exertion or rest, hypoxia, and excessive sputum production.

The polypeptides of the invention can be used alone or together with other treatments for COPD, including, bronchodilators, antibiotics, and oral or intravenous steroids.

In other embodiments, the methods of the invention are used to treat or limit the incidence of inflammation. As used herein, inflammation means the response of the immune system to infection, irritation, or associated with foreign bodies (introduction of biomaterials) in the body.

Symptoms characteristic of inflammation (for which the methods of the invention can be used to treat or reduce incidence of) include, but are not limited to redness, heat, swelling, pain, and dysfunction of the organs involved. Specific inflammatory disorders that can be treated, or whose incidence can be reduced, by the methods of the invention include, but are not limited to, asthma, arthritis (rheumatoid or degenerative), sepsis, endotoxemic shock, psoriasis, radiation enteritis, scleroderma, cirrhosis, interstitial fibrosis, Chrohn's disease, appendicitis, gastritis, laryngitis, meningitis, pancreatitis, and otitsis.

While not being bound by any specific mechanism of action, the inventors believe that administration of the polypeptides of the invention to a patient in need of anti-inflammatory treatment suppresses the response to inflammatory cytokines including but not limited to TGF β1.

In all of the above embodiments of the therapeutic methods of the invention, the polypeptides of the invention can be used as the sole active agent, or can be combined with one or more other treatments for the indication, as determined by an attending physician.

As used herein for all of the methods of the invention, an “amount effective” of the one or more polypeptides is an amount that is sufficient to provide the intended benefit of treatment. An effective amount of the polypeptides that can be employed ranges generally between about 0.01 μg/kg body weight and about 10 mg/kg body weight, preferably ranging between about 0.05 μg/kg and about 5 mg/kg body weight. However dosage levels are based on a variety of factors, including the type of injury, the age, weight, sex, medical condition of the individual, the severity of the condition, the route of administration, and the particular compound employed. Thus, the dosage regimen may vary widely, but can be determined routinely by a physician using standard methods.

The present invention may be better understood with reference to the accompanying examples that are intended for purposes of illustration only and should not be construed to limit the scope of the invention, as defined by the claims appended hereto.

### EXAMPLE 1

### HSP27 Kinase Inhibitor Peptide (BIP) Inhibits Phosphorylation of HSP27 In Vitro

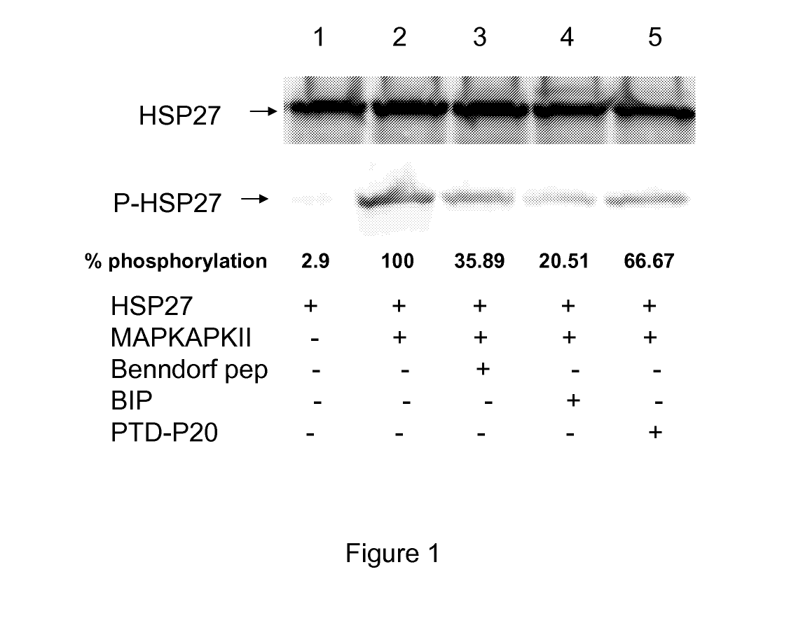

Recombinant HSP27 (11 g) was phosphorylated with no enzyme (control, lane 1), MAPKAPKII (50 ng) in the absence (lane 2) or presence of 200 μM (KKKALNRQLGVAA) (SEQ ID NO: 44) obtained from Calbiochem, (lane 3), or BIP (HSP27 kinase inhibitor peptide, WLRRIKAWLRRIKALNRQLGVAA (SEQ ID NO: 45) (lane 4) or HSP20 phosphopeptide (PTD-P20, lane 4) for 30 min at 30° C. The reaction mixtures were inactivated and separated by SDS-PAGE and transferred to PVDF membrane. The blot was probed with antibodies against HSP27 and phospho-HSP27. The ratio of P-P27 to P27 was calculated and the percent phosphorylation was determined with respect to the phosphorylation obtained in the absence of inhibitor (lane 2). The data is shown in FIG. 1. This blot is a representative of 2 separate experiments. These data demonstrate that BIP inhibits MAPKAPKII induced phosphorylation of HSP27 in vitro.

### EXAMPLE 2

### BIP Inhibits TGFβ1 Induced Expression of CTGF and Collagen

Chronic inflammation leading to fibrosis is but one mechanism that has recently received much attention. In affected tissues, chronic inflammation generally precedes fibrosis and inflammatory cell-derived cytokines are crucial mediators of fibrosis. Several cytokines have been identified as being critical in this process. One of these is transforming growth factor-β (TGF-β1). TGF-β1 is expressed at high levels during tissue remodeling and greatly affects the deposition of fibronectins and collagens via CTGF expression.

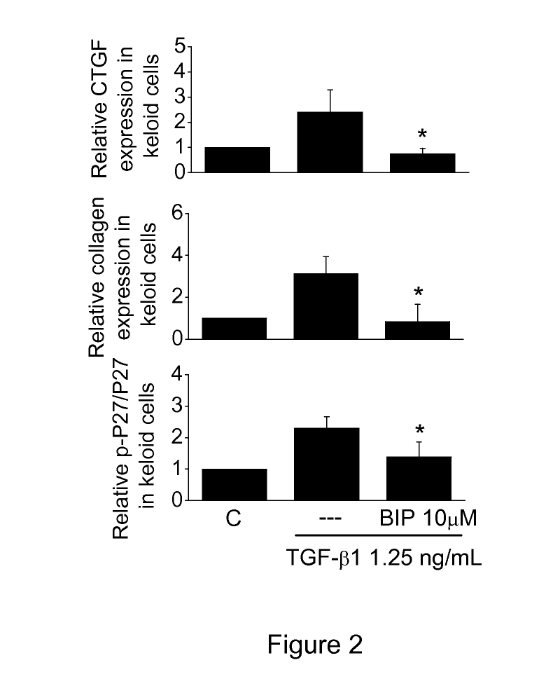

One important downstream signaling event in the TGFB signaling pathway is the activation of HSP27 kinase. We have designed a cell permeable inhibitor of this kinase (BIP) that effectively blocks its activity. In this study, we employ stable isotopic labeling by amino acids in cell culture (SILAC) as a means to quantitate the protein expression differences manifest during 1) TGF-β1 stimulation, 2) TGF-β1 stimulation in the presence of BIP, or 3) no treatment at all (control) in a keloid fibroblast cell line. Human keloid fibroblasts were grown to 80% confluence, and then serum-starved for 48 h. The cells were subsequently stimulated with nothing (control), with 1.25 ng/ml of transforming growth factor beta 1 (TGFβ) for 24 h, or 10 μM of a BIP for 2 h followed by TGFβ1 for 24 h (TGFβ+BIP). The expression of CTGF and collagen was assessed by immunoblot and normalized to b-actin expression. Phospho-HSP27 was expressed as a ratio to endogenous HSP27. Quantitative results represent average ±standard deviation from 3 independent experiments. The data is shown in FIG. 2 and demonstrate that BIP inhibits TGFβ1 induced expression of CTGF and collagen.

### EXAMPLE 3

### HSP27 Kinase Inhibitor Peptide (BIP) Inhibits TGF-β1 Induced Expression of CTGF and Collagen in Human Keloid Fibroblasts

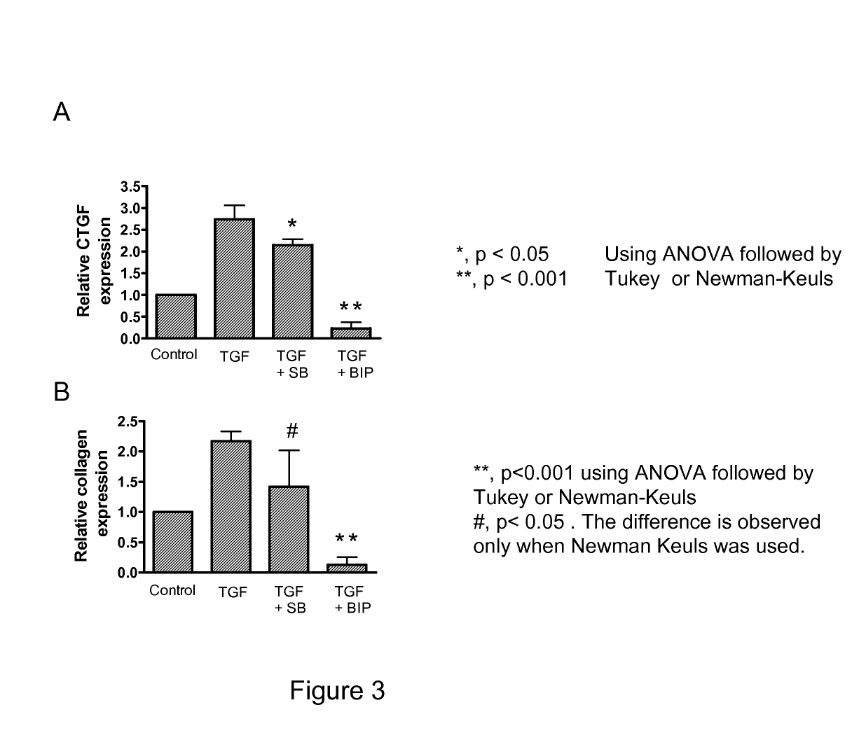

Human keloid fibroblasts were maintained in Dulbecco's modified Eagle's medium (DMEM) supplemented with fetal calf serum (10%) and streptomycin/penicillin (1%). They were grown to 80% confluence, and then serum-starved for 48 h. The cells were subsequently stimulated with nothing (control), with 1.25 ng/ml of transforming growth factor beta 1 (TGF-β1) for 24 h, 25 μM of a p38 MAP kinase inhibitor, SB 203580, for 2 h followed by TGF-β1 (TGF+SB) or 60 μM of test polypeptide WLRRIKAWLRRIKALNRQLGVAA (BIP) (SEQ ID NO: 45) for 2 h followed by TGF-β1 for 24 h (TGF+BIP). The expression of CTGF and collagen was assessed by immunoblot and normalized to GAPDH (loading control) expression. Results in the graphs (FIG. 3) represent average ±standard deviation from 3 independent experiments. These results demonstrate that BIP inhibits TGF-β1 induced expression of CTGF and collagen in human keloid fibroblasts.

In a further example, BIP was used at lower concentrations (5 or 10 μM). The resulting data showed that 10 μM, BIP inhibits TGF-β1 induced expression of CTGF and collagen.

### EXAMPLE 4

### HSP27 Kinase Inhibitor Peptide (BIP) Inhibits TGF-β1 Induced Stress Fiber Formation in Human Keloid Fibroblasts

Human keloid fibroblasts were maintained in Dulbecco's modified Eagle's medium (DMEM) supplemented with fetal calf serum (10%) and streptomycin/penicillin (1%). They were grown on cover slips and then serum-starved for 48 h. The cells were subsequently stimulated with nothing (control), with 1.25 ng/ml of transforming growth factor beta 1 (TGF-β1) for 24 h or 60 μM HSP27 kinase inhibitor peptide (BIP) for 2 h followed by TGF-β1 for 24 h (TGF b1+BIP). Cells were washed, processed for microscopy and labelled with Alexa 586-conjugated phalloidin to reveal the actin cytoskeleton and DAPI to reveal nucleus, magnification 40×. The data demonstrated that pre-treatment of cells with BIP followed by TGF b1 led to loss of central actin and reduced stress fiber formation in keloid fibroblasts.

### EXAMPLE 5

### HSP27 Kinase Inhibitor Peptide (BIP) Inhibits TGF-β1 Induced Phosphorylation of HSP27 in Human Keloid Fibroblasts (Fluorescence Microscopy)

Human keloid fibroblasts were maintained in Dulbecco's modified Eagle's medium (DMEM) supplemented with fetal calf serum (10%) and streptomycin/penicillin (1%). They were grown on cover slips and then serum-starved for 48 h. The cells were subsequently stimulated with nothing (control), with 1.25 ng/ml of transforming growth factor beta 1 (TGF-β1) for 24 h or 60 μM HSP27 kinase inhibitor peptide (BIP) for 2 h followed by TGF-β1 for 24 h (TGF b1+BIP). Cells were washed, processed for microscopy and labelled with Cy2 for phospho-HSP27 (ser 78/82, green fluorescence), Alexa 586-conjugated phalloidin (red) to reveal the actin cytoskeleton and DAPI (blue) to reveal nucleus, magnification 40×. Pre-treatment of cells with BIP followed by TGFb1 reduced phosphorylation of HSP27 in keloid fibroblasts. These data demonstrated that BIP inhibits TGF-β1 induced phosphorylation of HSP27 in human keloid fibroblasts.

### EXAMPLE 6

### HSP27 Kinase Inhibitor Peptide (BIP) Inhibits TGF-β1 Induced Phosphorylation of HSP27 in Human Keloid Fibroblasts (Immunoblot)

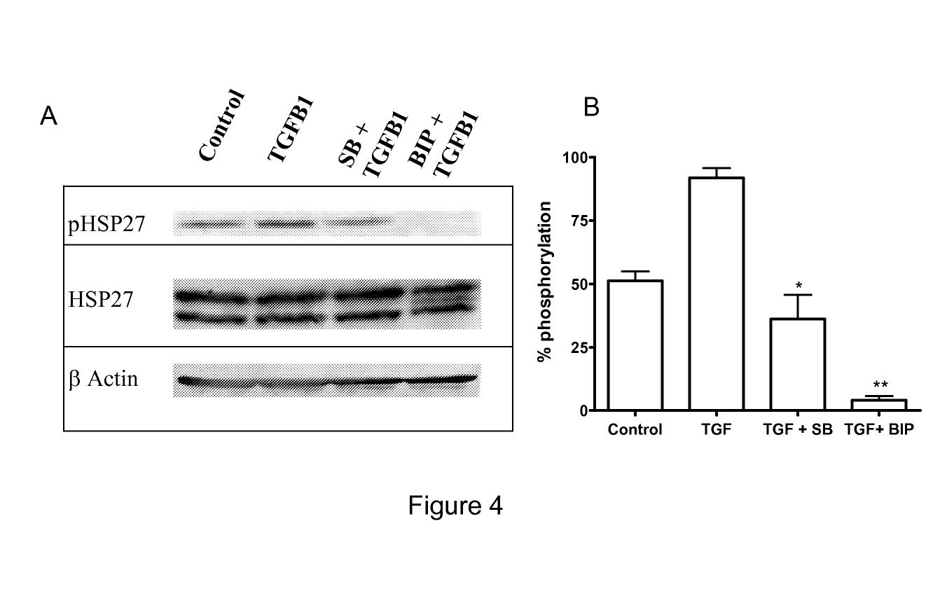

Human keloid fibroblasts were maintained in Dulbecco's modified Eagle's medium (DMEM) supplemented with fetal calf serum (10%) and streptomycin/penicillin (1%). They were grown to 80% confluence, and then serum-starved for 48 h. The data are shown in FIG. 4. A, The cells were subsequently stimulated with nothing (lane 1), with 1.5 ng/ml of Transforming growth factor beta 1 (TGF b1) for 24 h (Lane 2), 25 μM of a p38 MAP kinase inhibitor, SB 203580 for 2 h followed by TGFβ-1 (Lane 3) or 60 μM HSP27 kinase inhibitor peptide (BIP) for 2 h followed by TGFβ-1 for 24 h (Lane 4). The phosphorylation of HSP27 was assessed by immunoblot analysis using antibodies as indicated. B, The ratio of P-P27 to P27 was calculated and the percent phosphorylation was determined with respect to the phosphorylation obtained with TGF b (lane 2). These data further demonstrate that BIP effectively inhibited phosphorylation of HSP27, n=3, *, p<0.05; ** p<0.0001 using ANOVA followed by Tukey and Newman-Keuls.

In a separate example, lower concentrations of BIP (5 μM and 10 μM) were employed. Human keloid fibroblasts were serum-starved in DMEM medium containing 0.5% FBS for 48 h prior to treatment. TGF-β1-stimulated cells were treated with BIP (5 or 10 μM) for 24 h, and HSP27 and phospho-HSP27 levels were quantified relative to β-actin expression to correct for loading differences and normalized to untreated levels. The resulting data demonstrated that, under these assay conditions, 10 μM BIP inhibits TGF-β1 induced phosphorylation of HSP27 in human keloid fibroblasts.

### EXAMPLE 7

### The p38 MAP Kinase Inhibitor, SB 203580 and HSP27 Kinase Inhibitor, BIP, Enhances Sodium Nitroprusside Induced Relaxation of Saphenous Vein

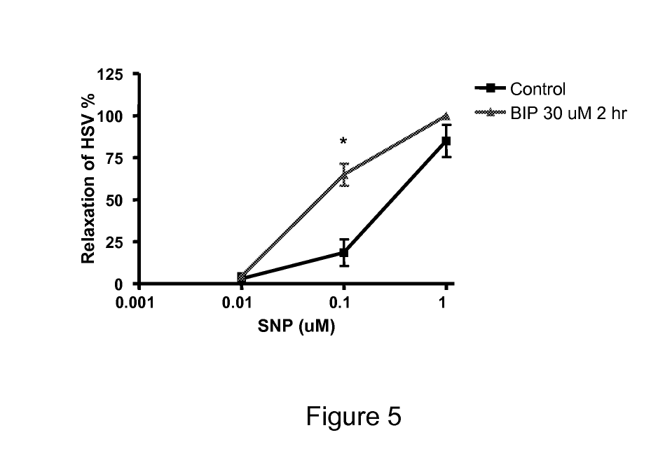

Segments of human saphenous vein (HSV) were equilibrated in a muscle bath and pretreated with buffer (control), 25 μM SB203580 (SB) or 30 μM BIP for 2 hrs and then contracted with norepinephrine (NE, 0.5 μM) and relaxed with increasing doses of sodium nitroprusside (SNP, 0-1 μM). The results are shown in FIG. 5. SB or BIP treatment led to significant increases in the relaxation of saphenous vein. * p=0.0335, # p=0.0108 at 0.1 μM SNP compared to control, n=3.

### EXAMPLE 8

### HSP27 Kinase Inhibitor Does Not Inhibit EC Proliferation

Human Aortic Endothelial Cells (Passage 2) (Cascade Biologics) were seeded into 96-well plates at a concentration of 2000 cells per well as determined using a hemocytometer and allowed to adhere for 4 hours. They were then treated with a final concentration of 1, 10 or 50 μM MK2 inhibitor peptide (BIP), 1, 10, or 50 μM scrambled peptide (SCR), or and equal volume of PBS. Cells were returned to the incubator for 16 hours and counted on a Molecular Devices M5 spectrophotometer after 1 hour treatment with CyQuant NF Cell Proliferation Assay (Invitrogen).

These data show that at doses where the polypeptides of the invention suppress CTGF and collagen deposition by fibroblasts, they do not inhibit EC proliferation, supporting the hypothesis that the polypeptides will be effective at inhibiting intimal hyperplasia and not inhibiting EC lining if delivered from grafts and stents.

### EXAMPLE 9

### BIP or SB Treatment Inhibits the Thickening of the Intimal Layer in Saphenous Vein

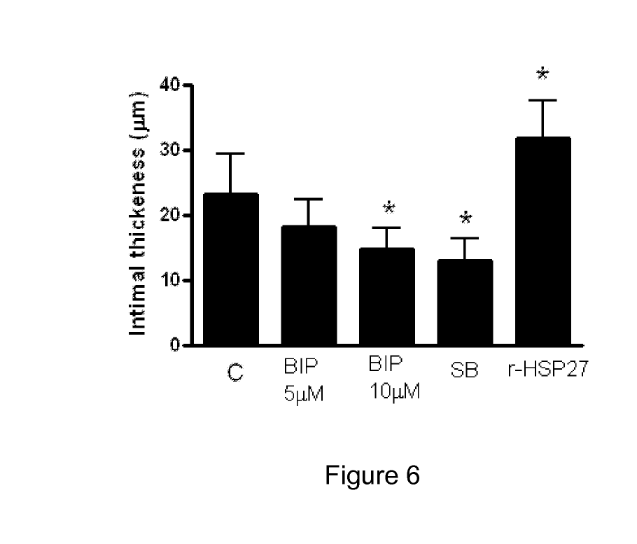

Human saphenous vein rings were cultured in RPMI medium supplemented with L-glutamine (1%), penicillin/streptomycin (1%) and FBS (30%) at 5% CO2 and 37° C. for 14 days. The rings were either untreated (C) or treated with BIP (5 or 10 μM), SB (20 μM) or recombinant HSP27 protein (r-HSP27). * p<0.05 compared to the control (untreated) rings. After 14 days, the rings were fixed in formalin, embedded in paraffin, sectioned at 10 micrometer and stained using Weigert's Resorcin Fuchsin. The slides were analyzed by Zeiss microscope with 40× objective. The intimal thickness was measured using Zeiss and Adobe Photoshop software. The data is shown in FIG. 6, and demonstrate that BIP treatment inhibits thickening of the intimal layer in saphenous vein.

### EXAMPLE 10

### BIP or SB Treatment Inhibits CTGF Expression and Phosphorylation of HSP27 in Saphenous Vein in an Organ Culture Model

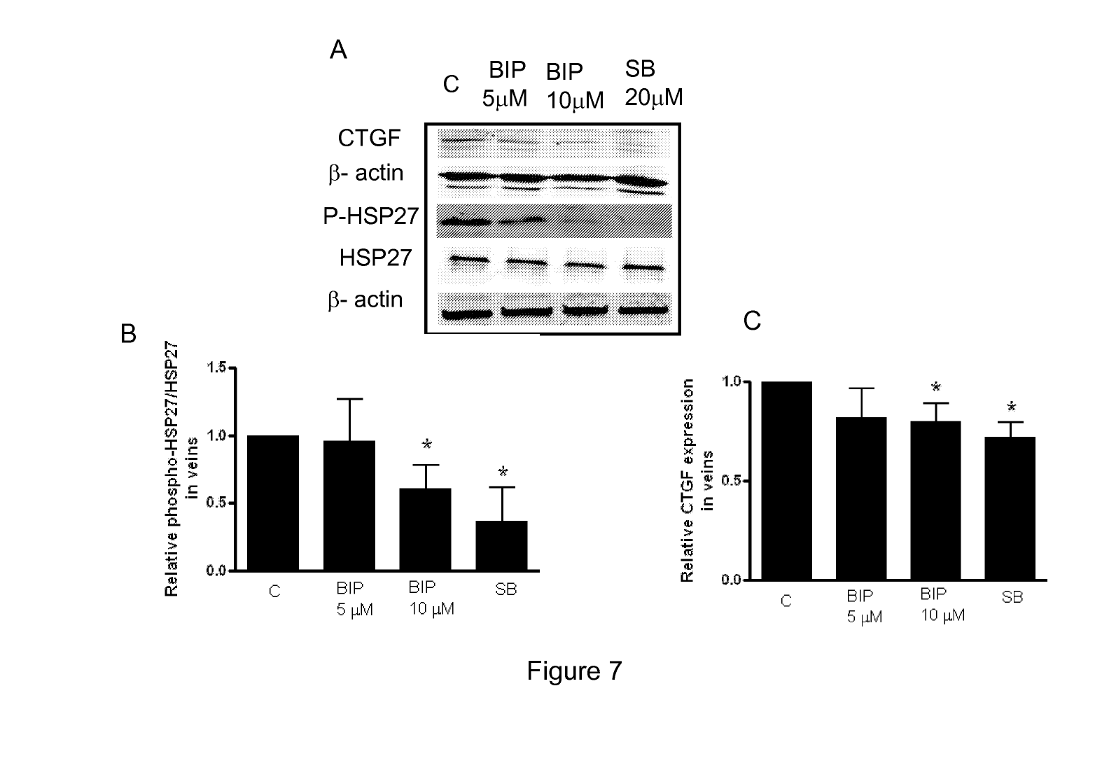

The human saphenous vein rings were cultured in RPMI medium supplemented with L-glutamine (1%), penicillin/streptomycin (1%) and FBS (30%) at 5% CO2 and 37° C. for 14 days. The rings were either untreated (Control, C) or treated with BIP (5 or 10 μM) or SB (20 μM). Rings were frozen after 14 days and protein was extracted and separated by SDS-PAGE and analyzed by western blot using the antibodies as indicated. The data is shown in FIG. 7; Panel A is a representative blot, and Panels B and C show the data summary. The bands were quantified by densitometry, and CTGF, phosphorylated HSP27 or non-phosphorylated HSP27 expressions were related to actin expression to correct for loading differences. The expression of CTGF and HSP27 and phospho-HSP27 in untreated rings was set to 1 for comparison of different blots. Data expressed as mean ±standard deviation of 6 experiments (panel right). *: p<0.05 compared to the untreated rings (C). The data demonstrate that BIP treatment inhibits CTGF expression and phosphorylation of HSP27 in saphenous vein in an organ culture model.

### EXAMPLE 11

### BIP or SB Treatment Inhibits Phosphorylation of HSP27 in Rat Aortic Smooth Muscle, A 7R5 Cells

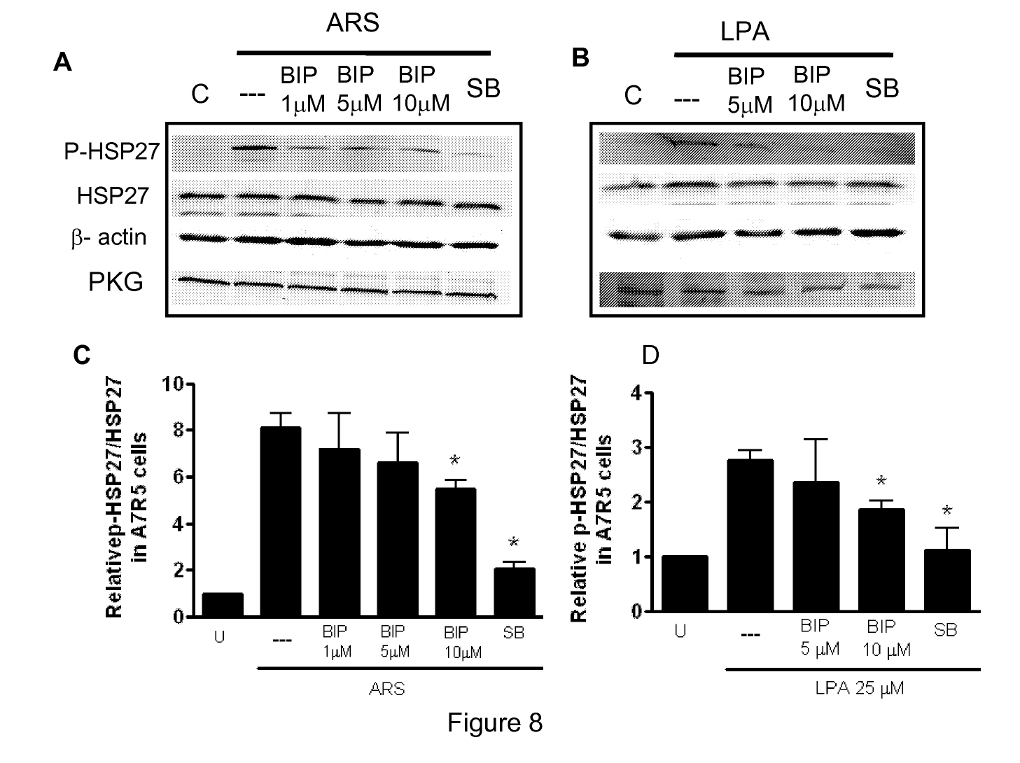

The cells were serum starved with DMEM medium supplemented with 1% BSA and 1% penicillin/streptomycin for 24 h. The phosphorylation of HSP27 was induced using arsenite (ARS, 500 μM) or lysophosphatidic acid (LPA, 25 μM) for 0.5 h. Cells were either untreated or pre-treated with BIP (1, 5 or 10 μM) or SB (20 μM) for 2 h prior to the stimulation with ARS or LPA. Cells were frozen after treatment and analyzed by SDS-PAGE and western blot. The data are shown in FIG. 8; Panels A and B are representative western blots, while Panels C and D provide the data. The bands were quantified by densitometry, and phosphorylated HSP27 or non-phosphorylated HSP27 expressions were related to actin expression to correct for loading differences. The ratio of phosphorylated HSP27/HSP27 (relative p-HSP27/HSP27) in untreated cells was set to 1 for comparison of different blots. Data expressed as mean ±standard deviation of 4 experiments. *: p<0.05 compared to the untreated cells (C). The data demonstrate that BIP treatment inhibits phosphorylation of HSP27 in Rat aortic smooth muscle, A7R5 cells.

### EXAMPLE 12

### BIP or SB Treatment Inhibits LPA Induced CTGF Expression in Rat Aortic Smooth Muscle (A 7R5) Cells

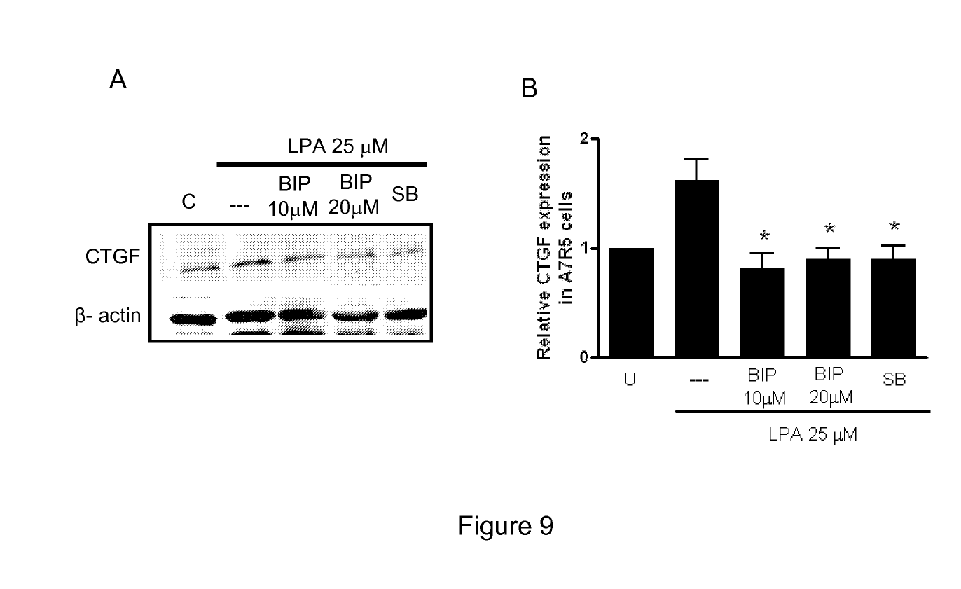

The cells were serum starved with DMEM medium supplemented with 1% BSA and 1% penicillin/streptomycin for 24 h. Cells were either untreated (C) or pre-treated with BIP (10 or 20 μM) or SB (20 μM) for 2 h prior the stimulation with lysophosphatidic acid (LPA, 25 μM). After 24 h the cells were lysed, protein extracted and separated by SDS-PAGE and analyzed by western blotting. The data are shown in FIG. 9; Panel A is a representative western blot, while Panel B provides the data. The bands were quantified by densitometry, and CTGF expression was related to actin expression to correct for loading differences. CTGF expression in untreated cells (C) was set to 1 for comparison of different blots. Data expressed as mean ±standard deviation of 3 experiments. *: p<0.05 compared to the cells stimulated with LPA. The data demonstrate that BIP treatment inhibits LPA induced CTGF expression in rat aortic smooth muscle (A7R5) cells.

### EXAMPLE 13

### BIP Inhibits Migration of Rat Aortic Smooth Muscle (A 7R5) Cells

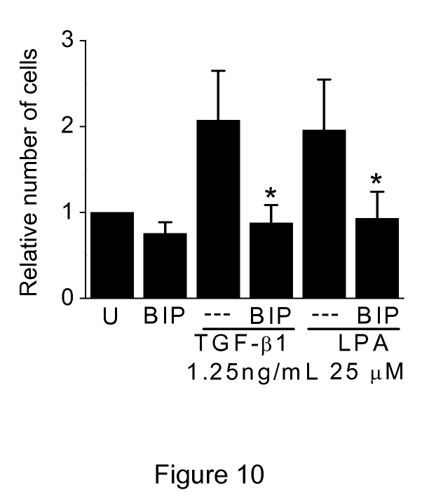

A7R5 cells were serum starved with DMEM medium supplemented with 1% BSA and 1% penicillin/streptomycin for 24 h. A scratch made in the dishes using the tip of a pipette, and the cells were rinsed 3 times with PBS. Cells were untreated (C) or pre-treated with BIP (10 μM) for 2 h prior the stimulation with lysophosphatidic acid (LPA, 25 μM) or TGF (2.5 ng/mL). *: p<0.05 compared to the cells stimulated with LPA or TGF. The data are shown in FIG. 10 and demonstrate that BIP inhibits migration of rat aortic smooth muscle (A7R5) cells

### EXAMPLE 14

### HSP27 Phosphorylation is Increased in Blood Vessels from Hypertensive Patients

Remnant strips of human saphenous vein after coronary or peripheral vascular reconstruction were hung in a muscle bath as described in preceding examples, and the strips were precontracted with norepinephrine and relaxed with sodium nitroprusside. The data demonstrated an inverse correlation between the relative amount of relaxation and systolic blood pressure (n=13 patients, p<0.05). The data further showed an inverse correlation between the % relaxation response to SNP and the amount of phosphorylated HSP27 determined with immunoblotting (p<0.05). Thus, these data demonstrate that HSP27 phosphorylation is increased in blood vessels from hypertensive patients, and indicates that such patients would benefit from the treatment methods of the invention.

We claim:

1. A polypeptide comprising a sequence according to general formula I

| | |
|---|---|
| Z1-X1-LNRQLGVAA-Z2 | (SEQ ID NO:1) |

wherein Z1 and Z2 are independently absent or are transduction domains; and
X1 is selected from the group consisting of KA, KKA, and KKKA (SEQ ID NO: 46), or is absent, with the proviso that if X1 is KKKA (SEQ ID NO: 46), then at least one of Z1 and Z2 is a transduction domain, and wherein when X1 is absent, then Z1 is a transduction domain ending in KA.

2. The polypeptide of claim 1, wherein X1 is absent, and Z1 is a transduction domain ending in KA.

3. The polypeptide of claim 1 wherein the polypeptide comprises

| | |
|---|---|
| WLRRIKAWLRRIKALNRQLGVAA. | (SEQ ID NO:45) |

4. The polypeptide of claim 1 wherein the polypeptide consists of

| | |
|---|---|
| WLRRIKAWLRRIKALNRQLGVAA. | (SEQ ID NO:45) |

5. A composition comprising the polypeptide of claim 1 and a pharmaceutically acceptable carrier.

6. A composition comprising the polypeptide of claim 3 and a pharmaceutically acceptable carrier.

7. An isolated nucleic acid sequence encoding the polypeptide of claim 1.

8. An isolated nucleic acid sequence encoding the polypeptide of claim 3.

9. A recombinant expression vector comprising the isolated nucleic acid of claim 7.

10. A recombinant expression vector comprising the isolated nucleic acid of claim 8.

11. A host cell transfected with the recombinant expression vector of claim 9.

12. A host cell transfected with the recombinant expression vector of claim 10.

13. A biomedical device comprising one or more polypeptides according to claim 1.

14. A biomedical device comprising one or more polypeptides according to claim 3.

15. The biomedical device of claim 13 wherein the one or more polypeptides are disposed on or in a heparin coating.

16. The biomedical device of claim 14 wherein the one or more polypeptides are disposed on or in a heparin coating.

17. A method for one or more of the following therapeutic uses
(a) reducing smooth muscle cell proliferation and/or migration; (b) promoting smooth muscle relaxation; (c) increasing the contractile rate in heart muscle; (d) increasing the rate of heart muscle relaxation; (e) promoting wound healing; (f) treating and/or reducing fibrotic disorders and/or keloids; (g) reducing scar formation; (h) disrupting focal adhesions; (i) regulating actin polymerization; and (j) treating or reducing incidence of one or more of intimal hyperplasia, stenosis, restenosis, atherosclerosis, smooth muscle cell tumors and metastasis, smooth muscle spasm, angina, Prinzmetal's angina, ischemia, stroke, bradycardia, hypertension, cardiac hypertrophy, renal failure, stroke, pulmonary hypertension, asthma, toxemia of pregnancy, pre-term labor, pre-eclampsia/eclampsia, Raynaud's disease or phenomenon, hemolytic-uremia, non-occlusive mesenteric ischemia, anal fissure, achalasia, impotence, migraine, ischemic muscle injury associated with smooth muscle spasm, vasculopathy, bradyarrythmia, bradycardia, congestive heart failure, stunned myocardium, pulmonary hypertension, diastolic dysfunction, gliosis; chronic obstructive pulmonary disease, osteopenia, endothelial dysfunction, inflammation, rheumatoid arthritis, degenerative arthritis, sepsis, endotoxemic shock, psoriasis, radiation enteritis, scleroderma, cirrhosis, interstitial fibrosis, Chrohn's disease, appendicitis, gastritis, laryngitis, meningitis, pancreatitis, otitsis, and reperfusion injury;
wherein the method comprises administering to a subject in need thereof an effective amount to carry out the one or more therapeutic uses of a polypeptide according to claim 1.

18. A method for one or more of the following therapeutic uses
(a) reducing smooth muscle cell proliferation and/or migration; (b) promoting smooth muscle relaxation; (c) increasing the contractile rate in heart muscle; (d) increasing the rate of heart muscle relaxation; (e) promoting wound healing; (f) treating and/or reducing fibrotic disorders and/or keloids; (g) reducing scar formation; (h) disrupting focal adhesions; (i) regulating actin polymerization; and (j) treating or reducing incidence of one or more of intimal hyperplasia, stenosis, restenosis, atherosclerosis, smooth muscle cell tumors and metastasis, smooth muscle spasm, angina, Prinzmetal's angina, ischemia, stroke, bradycardia, hypertension, cardiac hypertrophy, renal failure, stroke, pulmonary hypertension, asthma, toxemia of pregnancy, pre-term labor, pre-eclampsia/eclampsia, Raynaud's disease or phenomenon, hemolytic-uremia, non-occlusive mesenteric ischemia, anal fissure, achalasia, impotence, migraine, ischemic muscle injury associated with smooth muscle spasm, vasculopathy, bradyarrythmia, bradycardia, congestive heart failure, stunned myocardium, pulmonary hypertension, diastolic dysfunction, gliosis; chronic obstructive pulmonary disease, osteopenia, endothelial dysfunction, inflammation, rheumatoid arthritis, degenerative arthritis, sepsis, endotoxemic shock, psoriasis, radiation enteritis, scleroderma, cirrhosis, interstitial fibrosis, Chrohn's disease, appendicitis, gastritis, laryngitis, meningitis, pancreatitis, and otitsis;
wherein the method comprises administering to a subject in need thereof an effective amount to carry out the one or more therapeutic uses of a polypeptide according to claim 3.

19. The method of claim 17 wherein the method is used to treat or reduce incidence of intimal hyperplasia.

20. The method of claim 18 wherein the method is used to treat or reduce incidence of intimal hyperplasia.

21. The method of claim 17 wherein the method is used to treat and/or reduce fibrotic disorders and/or keloids.

22. The method of claim 18 wherein the method is used to treat and/or reduce fibrotic disorders and/or keloids.

23. The method of claim 21, wherein the subject is of Asian or African descent.

24. The method of claim 22, wherein the subject is of Asian or African descent.

25. The method of claim 17 wherein the subject is suffering from or at risk of one or more of diabetic nephropathy, glomerulosclerosis, IgA nephropathy, diabetic retinopathy, macular degeneration, cirrhosis, biliary atresia, congestive heart failure, scleroderma, and abdominal adhesions.

26. The method of claim 18 wherein the subject is suffering from or at risk of one or more of diabetic nephropathy, glomerulosclerosis, IgA nephropathy, diabetic retinopathy, macular degeneration, cirrhosis, biliary atresia, congestive heart failure, scleroderma, and abdominal adhesions.

27. The method of claim 17, wherein the subject has an elevated level of one or more of the following biomarkers in a target tissue:
TGFβ1 expression;
collagen I expression;
CTGF expression; and
α-smooth muscle actin expression.

28. The method of claim 18, wherein the subject has an elevated level of one or more of the following biomarkers in a target tissue:
TGFβ1 expression;
collagen I expression;
CTGF expression; and
α-smooth muscle actin expression.

29. The method of claim 17 wherein the method is used to treat or reduce hypertension.

30. The method of claim 18 wherein the method is used to treat or reduce hypertension.


SEQUENCE LISTING (46 sequences)

SEQ ID NO: 1 - protein, 15 aa, Artificial
  Synthetic
XXXXXLNRQLGVAAX

SEQ ID NO: 2 - protein, 4 aa, Artificial
  Synthetic
XXXX

SEQ ID NO: 3 - protein, 9 aa, Artificial
  Synthetic
RRRRRRRRR

SEQ ID NO: 4 - protein, 13 aa, Artificial
  Synthetic
GRKKRRQRRRPPQ

SEQ ID NO: 5 - protein, 8 aa, Artificial
  Synthetic
RQRRKKRG

SEQ ID NO: 6 - protein, 8 aa, Artificial
  Synthetic
GRKKRRQR

SEQ ID NO: 7 - protein, 12 aa, Artificial
  Synthetic
AYARAAARQARA

SEQ ID NO: 8 - protein, 34 aa, Artificial
  Synthetic
DAATATRGRSAASRPTERPRAPARSASRPRRPVE

SEQ ID NO: 9 - protein, 27 aa, Artificial
  Synthetic
GWTLNSAGYLLGLINLKALAALAKKIL

SEQ ID NO: 10 - protein, 12 aa, Artificial
  Synthetic
PLSSIFSRIGDP

SEQ ID NO: 11 - protein, 16 aa, Artificial
  Synthetic
AAVALLPAVLLALLAP

SEQ ID NO: 12 - protein, 12 aa, Artificial
  Synthetic
AAVLLPVLLAAP

SEQ ID NO: 13 - protein, 15 aa, Artificial
  Synthetic
VTVLALGALAGVGVG

SEQ ID NO: 14 - protein, 21 aa, Artificial
  Synthetic
GALFLGWLGAAGSTMGAWSQP

SEQ ID NO: 15 - protein, 27 aa, Artificial
  Synthetic
GWTLNSAGYLLGLINLKALAALAKKIL

SEQ ID NO: 16 - protein, 18 aa, Artificial
  Synthetic
KLALKLALKALKAALKLA

SEQ ID NO: 17 - protein, 21 aa, Artificial
  Synthetic
KETWWETWWTEWSQPKKKRKV

SEQ ID NO: 18 - protein, 14 aa, Artificial
  Synthetic
KAFAKLAARLYRKA

SEQ ID NO: 19 - protein, 14 aa, Artificial
  Synthetic
KAFAKLAARLYRAA

SEQ ID NO: 20 - protein, 14 aa, Artificial
  Synthetic
AAFAKLAARLYRKA

SEQ ID NO: 21 - protein, 14 aa, Artificial
  Synthetic
KAFAALAARLYRKA

SEQ ID NO: 22 - protein, 16 aa, Artificial
  Synthetic
KAFAKLAARLYRKAGC

SEQ ID NO: 23 - protein, 16 aa, Artificial
  Synthetic
KAFAKLAARLYRAAGC

SEQ ID NO: 24 - protein, 16 aa, Artificial
  Synthetic
AAFAKLAARLYRKAGC

SEQ ID NO: 25 - protein, 16 aa, Artificial
  Synthetic
KAFAALAARLYRKAGC

SEQ ID NO: 26 - protein, 16 aa, Artificial
  Synthetic
KAFAKLAAQLYRKAGC

SEQ ID NO: 27 - protein, 16 aa, Artificial
  Synthetic
AGGGGYGRKKRRQRRR

SEQ ID NO: 28 - protein, 11 aa, Artificial
  Synthetic
YARAAARQARA

SEQ ID NO: 29 - protein, 11 aa, Artificial
  Synthetic
YGRKKRRQRRR

SEQ ID NO: 30 - protein, 14 aa, Artificial
  Synthetic
WLRRIKAWLRRIKA

SEQ ID NO: 31 - protein, 21 aa, Artificial
  Synthetic
WLRRIKAWLRRIKAWLRRIKA

SEQ ID NO: 32 - protein, 22 aa, Artificial
  Synthetic
YARAAARQARAKALNRQLGVAA

SEQ ID NO: 33 - protein, 22 aa, Artificial
  Synthetic
YGRKKRRQRRRKALNRQLGVAA

SEQ ID NO: 34 - protein, 19 aa, Artificial
  Synthetic
RQRRKKRGKALNRQLGVAA

SEQ ID NO: 35 - protein, 19 aa, Artificial
  Synthetic
GRKKRRQRKALNRQLGVAA

SEQ ID NO: 36 - protein, 25 aa, Artificial
  Synthetic
WLRRIKAWLRRIKAKALNRQLGVAA

SEQ ID NO: 37 - protein, 32 aa, Artificial
  Synthetic
WLRRIKAWLRRIKAWLRRIKAKALNRQLGVAA

SEQ ID NO: 38 - protein, 24 aa, Artificial
  Synthetic
YARAAARQARAKKKALNRQLGVAA

SEQ ID NO: 39 - protein, 24 aa, Artificial
  Synthetic
YGRKKRRQRRRKKKALNRQLGVAA

SEQ ID NO: 40 - protein, 21 aa, Artificial
  Synthetic
RQRRKKRGKKKALNRQLGVAA

SEQ ID NO: 41 - protein, 21 aa, Artificial
  Synthetic
GRKKRRQRKKKALNRQLGVAA

SEQ ID NO: 42 - protein, 27 aa, Artificial
  Synthetic
WLRRIKAWLRRIKAKKKALNRQLGVAA

SEQ ID NO: 43 - protein, 34 aa, Artificial
  Synthetic
WLRRIKAWLRRIKAWLRRIKAKKKALNRQLGVAA

SEQ ID NO: 44 - protein, 13 aa, Artificial
  Synthetic
KKKALNRQLGVAA

SEQ ID NO: 45 - protein, 23 aa, Artificial
  Synthetic
WLRRIKAWLRRIKALNRQLGVAA

SEQ ID NO: 46 - protein, 4 aa, Artificial
  Synthetic
KKKA